(19) United States
(12) Patent Application Publication
(10) Pub. No.: US 2013/0046268 A1
(43) Pub. Date: Feb. 21, 2013

(54) Waist Adhering Material for Paper Diaper

(71) Applicants: Po-An Tsai, Kaohsiung City (TW); Charng-Ching Ou, Taipei City (TW); Hsu-Feng Shih, Changhua County (TW)

(72) Inventors: Po-An Tsai, Kaohsiung City (TW); Charng-Ching Ou, Taipei City (TW); Hsu-Feng Shih, Changhua County (TW)

(21) Appl. No.: 13/561,038

(22) Filed: Jul. 29, 2012

(30) Foreign Application Priority Data
Aug. 19, 2011 (TW) M417905
Sep. 1, 2011 (TW) M422407

(51) Int. Cl. A61F 13/62

(52) U.S. Cl. 604/391

(57) ABSTRACT

A low-cost waist adhering material for paper diaper for mass production and easy application comprises a replacement material. The replacement material is composed of a non-textile fabric layer disposed at a top end for connecting with hooks of a paper diaper, and a molded positioning layer disposed underneath the non-textile fabric layer and on a surface of the paper diaper for positioning the non-textile fabric layer. The non-textile fabric layer has at least one through hole area. The through hole area is composed of a plurality of through holes spaced at intervals penetrating through the non-textile fabric layer.

### BACKGROUND OF THE INVENTION

1. Field of Invention

The present invention relates to a waist adhering material for paper diaper and more particularly to a replacement material for a loop of paper diaper.

2. Related Art

Currently, there are generally two methods for fixing a paper diaper:

Firstly, a traditional waist adhering tape used with a film made of polypropylene or polyethylene or polyethylene glycol terephthalate as a target area. The adhering tape is adhered on the polypropylene film to achieve the purpose of fixing. Its advantage is that the materials used are low cost.

Its drawbacks are that, if the user's hands have excessive water vapor or talcum powder or baby lotion and the adhering tape or polyethylene film is covered by it, the adhering effect and fixing effect will be reduced greatly or disappeared totally. The paper diaper will have to be disposed because it cannot be fixed anymore. Because the leakage proof base film of the paper diaper is made of polyethylene, the adhering tape will be very difficult to be pulled apart from the polyethylene film if the tape is adhered on the film carelessly. The surface of the polyethylene film can be damaged easily because of the pulling force.

Secondly, the hook system which employs hooks and a material made of polypropylene or polyethylene or a loop with meshed fabric as a target area. The way they are fixed together around the waist is like the first method mentioned previously. The hooks are adhered on the limited loop area. The advantage is that, even if the user's hands have excessive water vapor or talcum powder or baby lotion, the adhering or fixing effect is practically not affected.

However, the drawback of the hooks and loop is that, the materials used and product cost are relatively more expensive than the first method. Additionally, paper diaper is usually used one time before disposal which is even more environmentally unfriendly in comparing with the first method.

Therefore, improvements are made in the second method as a solution to the drawbacks of the first method, which make the first method more convenient for the users. However, the expensive cost of the hooks and loop method is the main reason of not able to replace the combination of adhering tape and polypropylene film entirely.

In view of the abovementioned, a replacement material for paper diaper of the present invention is provided to achieve the objects of reducing the cost and the amount of material used, enhancing the yield rate of production and economizing the limited resources of the earth.

### SUMMARY OF THE INVENTION

An object of the present invention is to provide a low-cost waist adhering material for paper diaper for mass production and easy application.

In order to solve the abovementioned problems and achieve the above-mentioned objects, the waist adhering material for paper diaper of the present invention providing two technical means:

A first technical means:

a waist adhering material for paper diaper, comprising:

a replacement material, characterized in that: the replacement material being composed of a non-textile fabric layer disposed at a top end for connecting with hooks of a paper diaper, and

a molded positioning layer disposed underneath the non-textile fabric layer and on a surface of the paper diaper for positioning the non-textile fabric layer;

the non-textile fabric layer having at least one through hole area, the through hole area being composed of a plurality of through holes spaced at intervals penetrating through the non-textile fabric layer.

According to the waist adhering material for paper diaper, the non-textile fabric layer and the molded positioning layer are either an elastic layer or a non-elastic layer or a combination of them; and

the elasticity coefficients of the non-textile fabric layer and the molded positioning layer can be different.

According to the waist adhering material for paper diaper, the through hole area is disposed in one of the shapes of: circular area, character area, strip area, rectangular area, pattern area, dot area, tartan area, regular striation area or irregular striation area.

A second technical means:

a waist adhering material for paper diaper, comprising:

a replacement material, characterized in that: the replacement material being composed of a non-textile fabric layer positioned on a surface of a paper diaper for connecting with hooks of the paper diaper;

the non-textile fabric layer having at least one through hole area, the through hole area being composed of a plurality of through holes spaced at intervals penetrating through the non-textile fabric layer.

According to the waist adhering material for paper diaper, the non-textile fabric layer is either an elastic layer or a non-elastic layer.

According to the waist adhering material for paper diaper, the through hole area is disposed in one of the shapes of: circular area, character area, strip area, rectangular area, pattern area, dot area, tartan area, regular striation area or irregular striation area.

Comparing with the effects of the conventional techniques:

1) The surface of the replacement material for paper diaper of the present invention is made more rough and uneven by the disposition of the through hole area on the non-textile fabric layer. Therefore, the non-textile fabric layer of the present invention can be more effectively cooperated with the hooks for fixing the paper diaper around the waist, and the conventional targeted loop area can be replaced.

2) In the present invention, the replacement material is composed of the non-textile fabric layer, or the non-textile fabric layer and the molded positioning layer. Therefore, the cost is a lot lower than the conventional loop. As a result, the overall cost of the paper diaper can be reduced.

The present invention will become more fully understood by reference to the following detailed description thereof when read in conjunction with the attached drawings.

### BRIEF DESCRIPTION OF THE DRAWINGS

FIG. 1 is a first perspective view of a waist adhering material for paper diaper used with a paper diaper according to a first embodiment of the present invention;

FIG. 2 is an exploded perspective view of FIG. 1;

FIG. 3 is a cross-sectional view of a replacement material of the waist adhering material for paper diaper of FIG. 1;

FIG. 4 is a perspective view of the waist adhering material for paper diaper of the present invention with hooks of the paper diaper being adhered on the replacement material;

FIG. 5 is a cross-sectional view of the waist adhering material for paper diaper of the first embodiment of the present invention with the hooks of the paper diaper being adhered on a non-textile fabric layer;

FIG. 6 is a second perspective view of the waist adhering material for paper diaper used with the paper diaper according to the first embodiment of the present invention;

FIG. 7 is a third perspective view of the waist adhering material for paper diaper used with the paper diaper according to the first embodiment of the present invention;

FIG. 8 is a perspective view of the waist adhering material for paper diaper used with the paper diaper according to a second embodiment of the present invention; and

FIG. 9 is an exploded perspective view of FIG. 8.

### DETAILED DESCRIPTION OF THE INVENTION

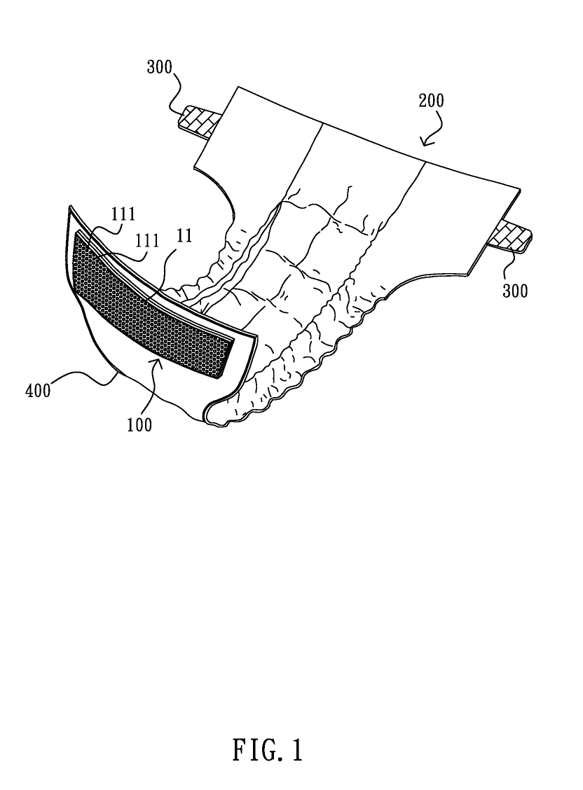

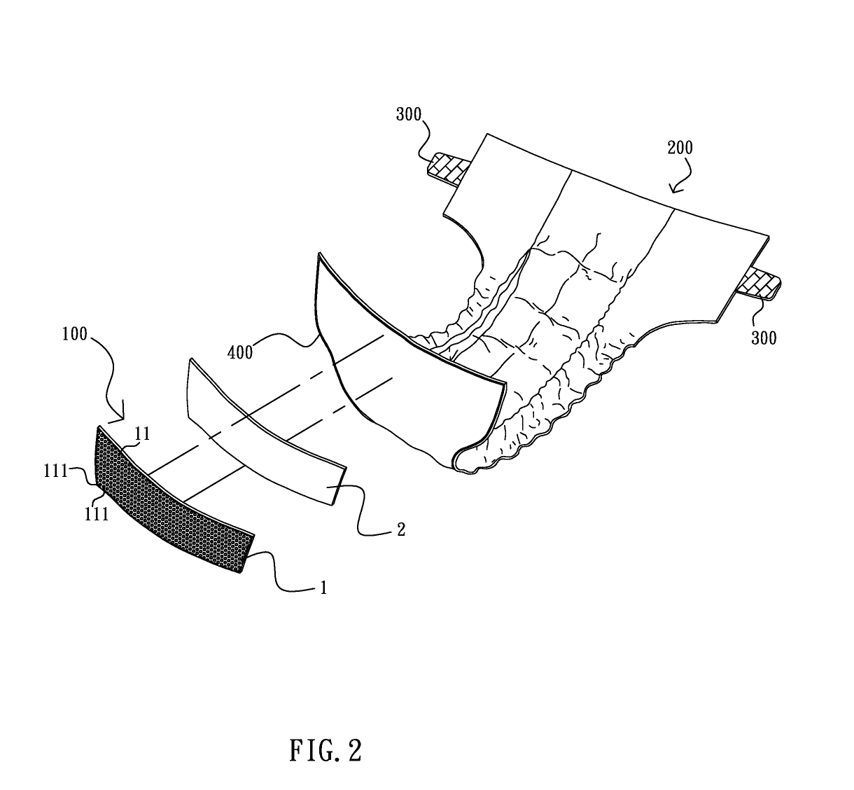

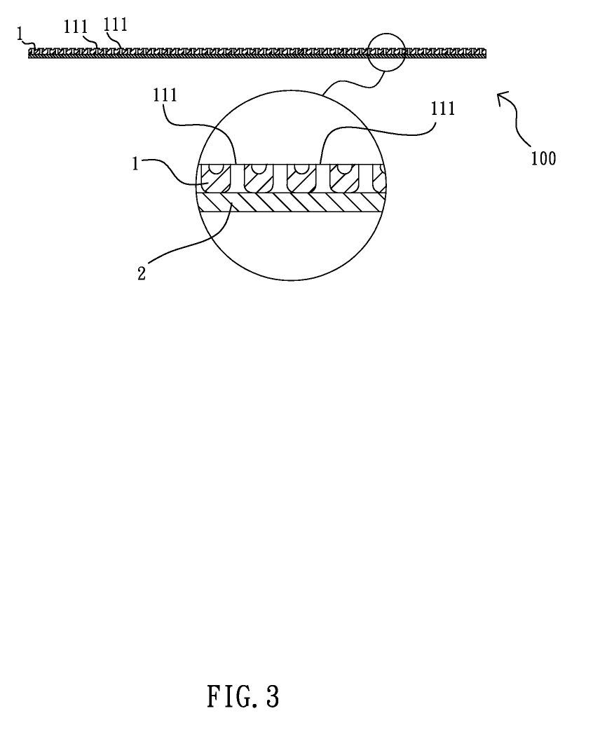

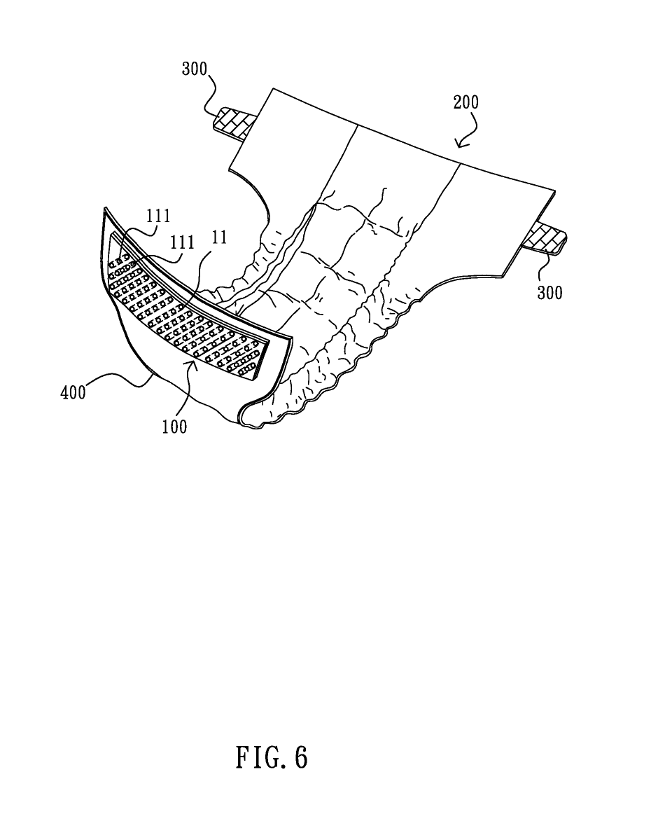

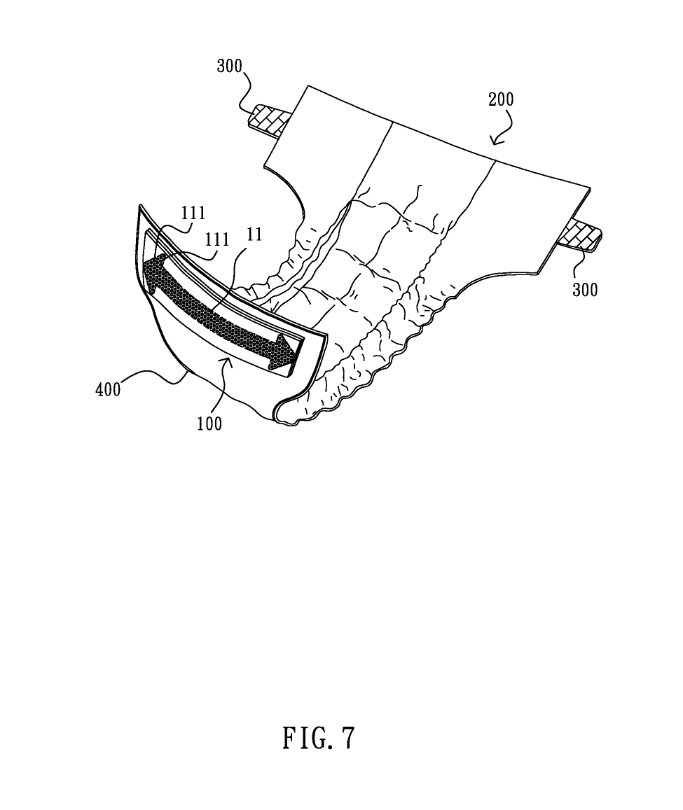

FIG. 1 is a first perspective view of a waist adhering material for paper diaper used with a paper diaper according to a first embodiment of the present invention; FIG. 2 is an exploded perspective view of FIG. 1; FIG. 3 is a cross-sectional view of a replacement material of the waist adhering material for paper diaper of FIG. 1; FIG. 6 is a second perspective view of the waist adhering material for paper diaper used with the paper diaper according to the first embodiment of the present invention; and FIG. 7 is a third perspective view of the waist adhering material for paper diaper used with the paper diaper according to the first embodiment of the present invention.

As shown in the drawings, the waist adhering material for paper diaper of the present invention, comprising:

a replacement material 100, characterized in that: the replacement material 100 being composed of a non-textile fabric layer 1 disposed at a top end for connecting with hooks 300 of a paper diaper 200, and

a molded positioning layer 2 disposed underneath the non-textile fabric layer 1 and on a surface 400 of the paper diaper 200 for positioning the non-textile fabric layer 1;

the non-textile fabric layer 1 having at least one through hole area 11, the through hole area 11 being composed of a plurality of through holes 111 spaced at intervals penetrating through the non-textile fabric layer 1.

Wherein, the surface of the replacement material 100 is made more rough and uneven by the disposition of the through hole area 11 on the non-textile fabric layer 1. Therefore, the non-textile fabric layer 1 replaces the conventional targeted loop area and can be more effectively cooperated with the hooks 300 for fixing the paper diaper 200 around the waist.

Additionally, the contact area of the non-textile fabric layer 1 is made three-dimensionally by the through holes 111 of the through hole area 11, and therefore the holding power between the non-textile fabric layer 1 and the molded positioning layer 2 is enhanced. Furthermore, the contact area between the non-textile fabric layer 1 and the hooks 300 can be increased in order to enhance the hooking or adhering effect. The non-textile fabric layer 1 with the through holes 111 is structurally similar to the conventional loop and therefore can be used repeatedly as an ideal replacement for the loop. The paper diaper 200 looks more artistic with the non-textile fabric layer 1 instead of the conventional loop.

Furthermore, the through holes 111 on the non-textile fabric layer 1 are formed by punching with needles. Adjustment on the quantity of the through holes 111 and the form or shape of the through holes 111 being presented can be achieved by adjusting the disposition of the needles. Therefore, the material of conventional loop can be replaced with the non-textile fabric layer 1 with the through holes 111 for mass production of the paper diapers. As a result, the production efficiency is enhanced, the cost of materials and the defective percentage can be reduced, the overall costs and the amount of materials used can be reduced and the emission of carbon can be reduced as well.

The cost of the non-textile fabric layer 1 is much lower than that of the fabric used for the loop. Therefore, the overall cost of the paper diaper 200 can be reduced as well. When the replacement material 100 is employed for the paper diaper 200, besides the convenience provided, the financial burden on consumers can also be lightened because of the lower price. As a result, the paper diaper 200 with the non-textile fabric layer 1 will become more popular amongst the consumers.

The non-textile fabric layer 1 and the molded positioning layer 2 are either an elastic layer or a non-elastic layer or a combination of them; and

the elasticity coefficients of the non-textile fabric layer 1 and the molded positioning layer 2 can be different.

Wherein, the selection of the non-textile fabric layer 1 and the molded positioning layer 2 is based on the characteristics of the paper diaper 200, and the application of the layers is based on the application needs of the paper diaper 200.

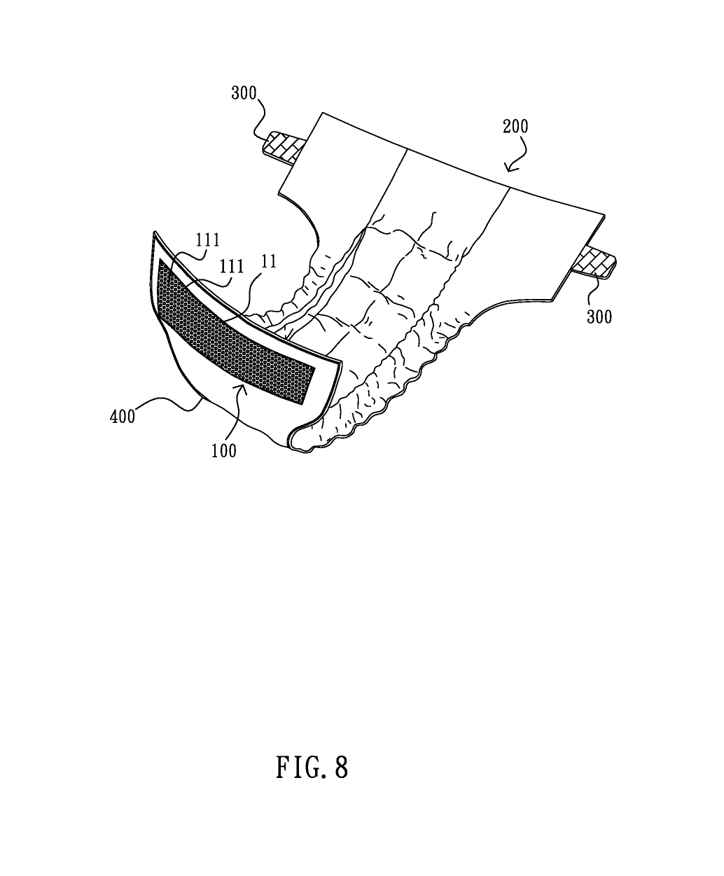

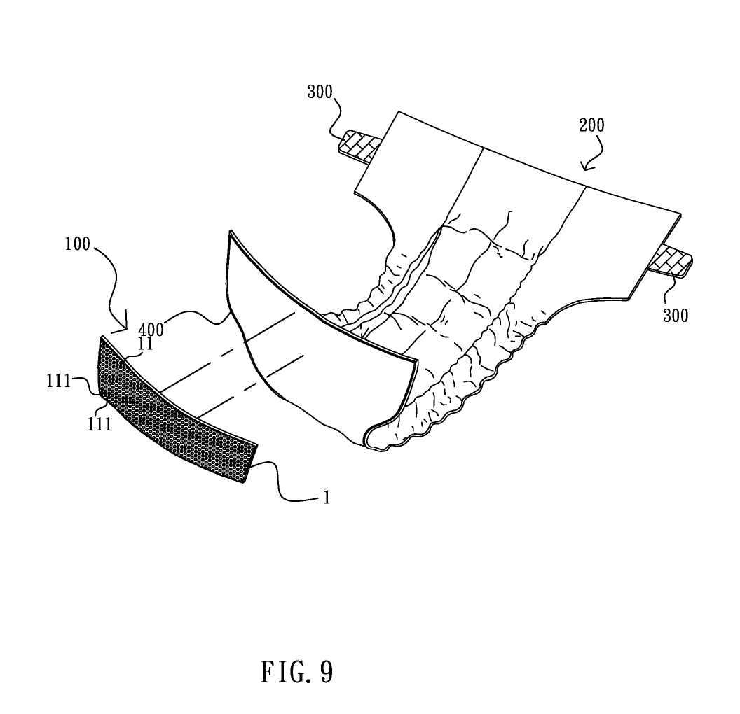

FIG. 8 is a perspective view of the waist adhering material for paper diaper used with the paper diaper according to a second embodiment of the present invention; and FIG. 9 is an exploded perspective view of FIG. 8.

As shown in the drawings, the waist adhering material for paper diaper of the present invention, comprising:

a replacement material 100, characterized in that: the replacement material 100 being composed of a non-textile fabric layer 1 positioned on a surface 400 of a paper diaper 200 for connecting with hooks 300 of the paper diaper 200;

the non-textile fabric layer 1 having at least one through hole area 11, the through hole area 11 being composed of a plurality of through holes 111 spaced at intervals penetrating through the non-textile fabric layer 1.

Wherein, the application of the non-textile fabric layer 1 can achieve the object of replacing the conventional loop provided that the overall effect is not affected. The non-textile fabric layer 1 is more suitable for using with the low-cost paper diaper 200 and the overall cost can therefore be reduced.

The non-textile fabric layer 1 is either an elastic layer or a non-elastic layer.

Wherein, the application of the elastic or non-elastic non-textile fabric layer 1 with the paper diaper 200 can avoid the problem of ineffectiveness of the replacement material 100.

In the two embodiments above, the through hole area 11 is disposed in one of the shapes of: circular area, character area, strip area, rectangular area, pattern area, dot area, tartan area, regular striation area or irregular striation area.

Wherein, the location and form or shape of the through hole area 11 of the waist adhering material for paper diaper applied in the paper diaper 200 are provided with different selections to meet the various requirements of the paper diaper 200 for adult or baby.

The look is more aesthetically pleasing by employing the through hole area 11 in different forms for matching different types of the paper diaper 200. Therefore, the replacement material 100 can have a wide rage of applications.

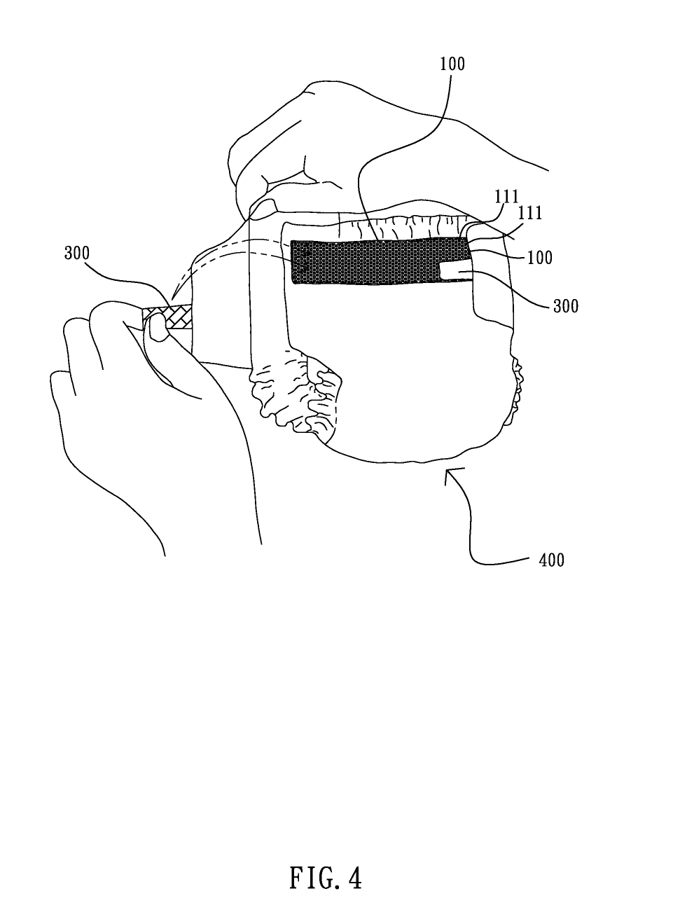

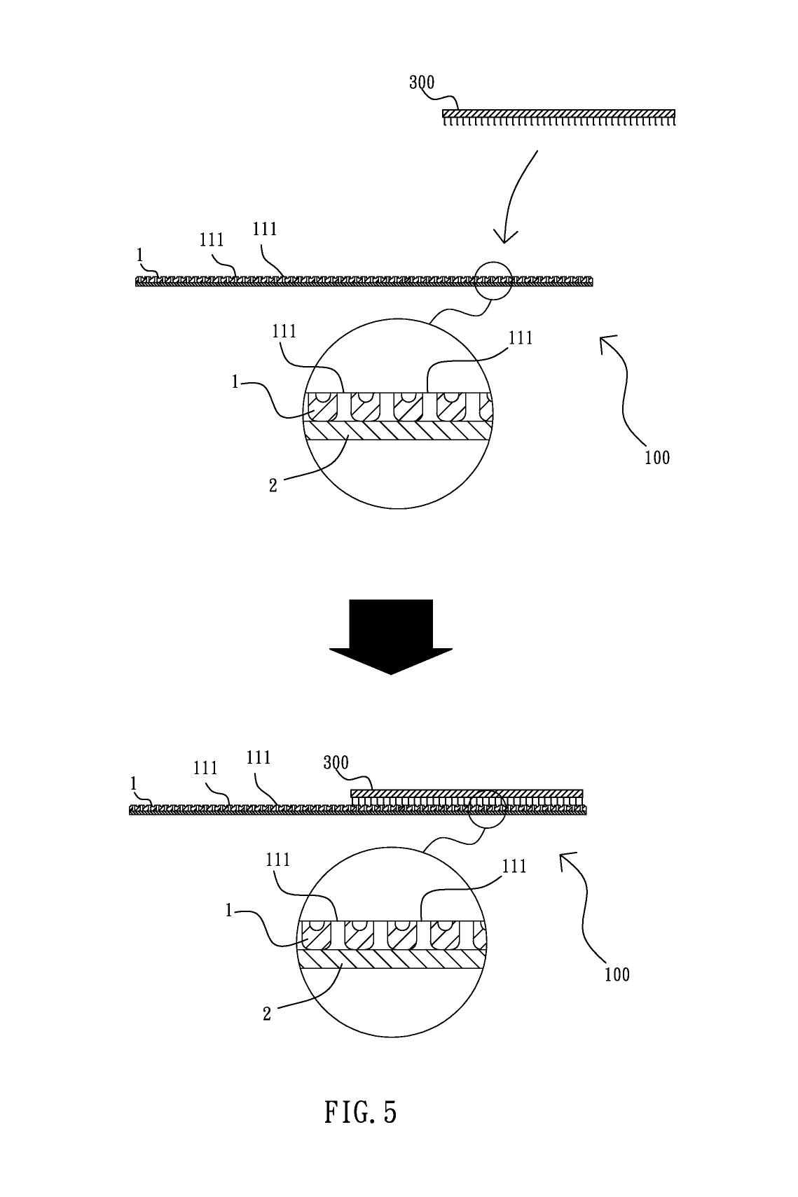

FIG. 4 is a perspective view of the waist adhering material for paper diaper of the present invention with the hooks of the paper diaper being adhered on the replacement material; and FIG. 5 is a cross-sectional view of the waist adhering material for paper diaper of the first embodiment of the present invention with the hooks of the paper diaper being adhered on the non-textile fabric layer.

As shown in the drawings, when the replacement material 100 of the present invention is applied on the paper diaper 200, besides that the overall costs and the amount of materials used can be reduced, the skin of the baby will not be adhered by the replacement material 100 which can be used repeatedly for convenience of checking. Even if skin moisturizing oil or water is spilled on the replacement material 100, the replacement material 100 and the hooks 300 can still be adhered together for fixing the paper diaper 200.

As a conclusion from the abovementioned, comparing the conventional loop with the non-textile fabric layer 1 or the cooperative combination of the non-textile fabric layer 1 and the molded positioning layer 2 with the application of the through hole area 11, the cost and the defective rate of the present invention are lower and the amount of materials used can be reduced. Furthermore, the present invention is more convenient to use for the consumers, and has better applicability, practicability and functionality for the manufacturers.

Note that the specifications relating to the above embodiments should be construed as exemplary rather than as limitative of the present invention, with many variations and modifications being readily attainable by a person of average skill in the art without departing from the spirit or scope thereof as defined by the appended claims and their legal equivalents.

What is claimed is:

1. A waist adhering material for paper diaper, comprising:
a replacement material, characterized in that: the replacement material being composed of a non-textile fabric layer disposed at a top end for connecting with hooks of a paper diaper, and
a molded positioning layer disposed underneath the non-textile fabric layer and on a surface of the paper diaper for positioning the non-textile fabric layer;
the non-textile fabric layer having at least one through hole area, the through hole area being composed of a plurality of through holes spaced at intervals penetrating through the non-textile fabric layer.

2. The waist adhering material for paper diaper as claimed in claim 1, characterized in that:
the non-textile fabric layer and the molded positioning layer are either an elastic layer or a non-elastic layer or a combination of them;
the elasticity coefficients of the non-textile fabric layer and the molded positioning layer can be different.

3. The waist adhering material for paper diaper as claimed in claim 1, characterized in that: the through hole area is disposed in one of the shapes of circular area, character area, strip area, rectangular area, pattern area, dot area, tartan area, regular striation area or irregular striation area.

4. A waist adhering material for paper diaper, comprising:
a replacement material, characterized in that: the replacement material being composed of a non-textile fabric layer positioned on a surface of a paper diaper for connecting with hooks of the paper diaper;
the non-textile fabric layer having at least one through hole area, the through hole area being composed of a plurality of through holes spaced at intervals penetrating through the non-textile fabric layer.

5. The waist adhering material for paper diaper as claimed in claim 4, characterized in that: the non-textile fabric layer is either an elastic layer or a non-elastic layer.

6. The waist adhering material for paper diaper as claimed in claim 4, characterized in that: the through hole area is disposed in one of the shapes of circular area, character area, strip area, rectangular area, pattern area, dot area, tartan area, regular striation area or irregular striation area.
